(19) United States
(12) Patent Application Publication
(10) Pub. No.: US 2024/0252737 A1
(43) Pub. Date: Aug. 1, 2024

(54) MEDICAL DEVICE PACKAGING

(71) Applicant: Teva Branded Pharmaceutical Products R&D, Inc., West Chester, PA (US)

(72) Inventors: Mark Destefano, West Chester, PA (US); Daniel Davenport, West Chester, PA (US)

(73) Assignee: Teva Branded Pharmaceutical Products R&D, Inc., West Chester, PA (US)

(21) Appl. No.: 18/560,811

(22) PCT Filed: Jun. 13, 2022
(86) PCT No.: PCT/US2022/033244

§ 371 Date: Nov. 14, 2023

(30) Foreign Application Priority Data
Jun. 14, 2021 (GB) 2108472.8

(51) Int. Cl. A61M 5/00; B65D 1/36; B65D 5/38; B65D 5/42; B65D 25/10; B65D 77/20

(52) U.S. Cl. CPC A61M 5/002; B65D 1/36; B65D 5/38; B65D 5/4208; B65D 5/4212; B65D 25/10; B65D 77/20; B65D 2203/00

(57) ABSTRACT

Medical device packaging comprising: a box comprising a first compartment and a second compartment, the first and second compartments being independently openable to reveal contents of the first and second compartments respectively; a cover moveable between a closed position and an open position and which, when placed in the closed position on the box, prevents the first and second compartments from being opened; instructions for use of the device provided on a foldable portion of the box, the instructions for use only being fully visible when the cover is placed in the open position and the foldable portion is moved from a folded to an unfolded position, wherein the first compartment is configured for storing a medicament vial or cartridge and the second compartment is configured for storing an injection device which may be an on body injector (“OBI”).

This disclosure relates to the field of medical device packaging, in particular to medical device packaging suitable for an injection device which may be an on-body injector (“OBI”).

### BACKGROUND

An on-body injector (“OBI”) is a medical device used to delivery larger doses of medicament than is generally possible with a syringe or autoinjector and/or where medicament needs to be delivered over a longer period of time. An OBI may be provided to the patient in a prefilled format, i.e., already containing the medicament. Alternatively, a vial or cartridge of liquid medicament is provided to the patient together with a sterile OBI and the patient or an associated caregiver assembles the OBI and medicament at the point of use.

Packaging is often the first interaction that a patient has with an OBI. Instructions for use (“IFU”) are provided to the patient as part of the packaging and may also include a Quick Reference Guide (“QRG”) comprising a simplified, often visually depicted, version of the IFU. Numerous steps are involved. It is imperative that the packaging is user-friendly and guides the patient through all of the steps involved in using their medical device properly. This is particularly important for OBIs where the patient may be less familiar with the use of the device and/or may use such devices infrequently. Generally, in the field of medical device packaging, human factors considerations and the user experience are critical. Any changes to such packaging must be very carefully considered and are non-trivial, since design-induced user errors may have significant safety implications.

### BRIEF SUMMARY OF THE DISCLOSURE

In accordance with an aspect of the present invention there is provided medical device packaging comprising:
a. a box comprising a first compartment and a second compartment, the first and second compartments being independently openable to reveal contents of the first and second compartments respectively;
b. a cover moveable between a closed position and an open position and which, when placed in the closed position on the box, prevents the first and second compartments from being opened;
c. instructions for use of the device provided on a foldable portion of the box, the instructions for use only being fully visible when the cover is placed in the open position and the foldable portion is moved from a folded to an unfolded position, wherein the first compartment is configured for storing a medicament vial or cartridge and the second compartment is configured for storing an injection device which may be an on-body injector (“OBI”).

By providing the instructions for use of the device on a foldable portion of the box, it is possible to guide the user sequentially through the steps of the IFU as each step or group of steps is sequentially revealed by unfolding the folded portion.

In an embodiment the foldable portion comprises at least two panels which are moveable from the folded to the unfolded position in a sequence such that steps of the instructions for use provided on each panel are revealed in a desired order. Preferably, said sequence includes opening said first compartment after moving one of said at least two panels from the folded position to the unfolded position. In an embodiment, said sequence includes opening said second compartment after moving a second one of said at least two panels from the folded position to the unfolded position.

In this way, the user is guided step by step through the desired sequence, with the steps of the IFU being revealed at the appropriate time before each of the independently openable first and second compartments are opened. This is preferable to the user being confronted with the entirety of the IFU before opening any of the compartments, risking the possibility that steps may be performed out of sequence, or steps may be missed. As mentioned above, design changes to medical device packaging are non-trivial in nature, given the potential safety implications for design-induced user error. Despite this, a technical advantage has been noted in the foldable portion of the box revealing only the relevant part of the IFU before each compartment is opened.

In an embodiment the foldable portion has an inner and an outer surface and at least one of said panels is provided on each of said inner and outer surfaces. This reduces the quantity of packaging material required.

The instructions for use may be printed directly onto said foldable portion. Alternatively, they may be printed onto stickers or the like permanently affixed to the foldable portion.

In an embodiment, the cover is a lid, moveable between said closed position and said open position. The cover may comprise said foldable portion.

In another embodiment, said cover is a removable sleeve or outer box, slidable over said first and second compartments between said open position and said closed position. The box may comprise a pull tab or pull loop to facilitate removal of the box from the removable sleeve or outer box.

Preferably, when the foldable portion is in the unfolded position, the foldable portion can be laid flat. This “laid-flat” configuration gives the user clarity that the packaging has been completely opened, thus improving user satisfaction.

In an embodiment, one or both of said first and second compartments comprises a removable or peelable cover and said compartments are independently openable by removal of said removable or peelable cover(s).

Said first compartment may further comprise an opening through which a medicament vial or cartridge can be inserted into said first compartment without opening said removable or peelable cover. This has the advantage that the medicament vial or cartridge may be stored separately from the rest of the packaging (e.g. refrigerated) until such appropriate time when it can be assembled into the packaging for supply to the end user.

According to another aspect of the invention there is provided a storage system for storing a medical device, for example, an on body injector (“OBI”), the storage system comprising
a base;
a tray mounted on the base and for receiving the medical device, and
a retainer for releasably retaining the medical device in the tray, wherein the retainer is moveable between a locked position in which the medical device cannot be removed from the tray and an unlocked position in which the medical device can be removed from the tray.

In an embodiment, the storage system further comprises a transfer mechanism capable of transferring medicament from a medicament vial or cartridge to the medical device, wherein the transfer mechanism comprises an unlocking mechanism which is actuable upon completion of medicament transfer to move the retainer from the locked to the unlocked position. By retaining the medical device in the tray, the user is prevented from removing the medical device from the tray early, for example before the device has been filled with medicament.

In an embodiment, the retainer is hingedly connected to the tray and, optionally, the transfer mechanism is permanently attached to the tray.

In an embodiment, the tray comprises a peripheral flange glued or otherwise permanently attached to the base. Preferably, the peripheral flange is sandwiched between two layers of the base so that, in use, the tray cannot be removed from the base.

### BRIEF DESCRIPTION OF THE DRAWINGS

Embodiments of the invention are further described hereinafter, by way of example only, with reference to the accompanying drawings, in which:

FIG. 1 is a perspective view of medical device packaging according to a first example embodiment of the present invention, showing the packaging in a fully closed position;

FIG. 2 shows the box removed from the cover/outer box and with the foldable portion in a fully unfolded position;

FIG. 2A is a cross-sectional view of part of the tray and the base card;

FIG. 3 shows the cover/outer box still in a closed position;

FIG. 4 shows the cover/outer box in an open position and the box removed from the cover/outer box, with the foldable portion in a folded position;

FIG. 5 shows the foldable portion in a partially unfolded position;

FIG. 6 shows the first compartment open;

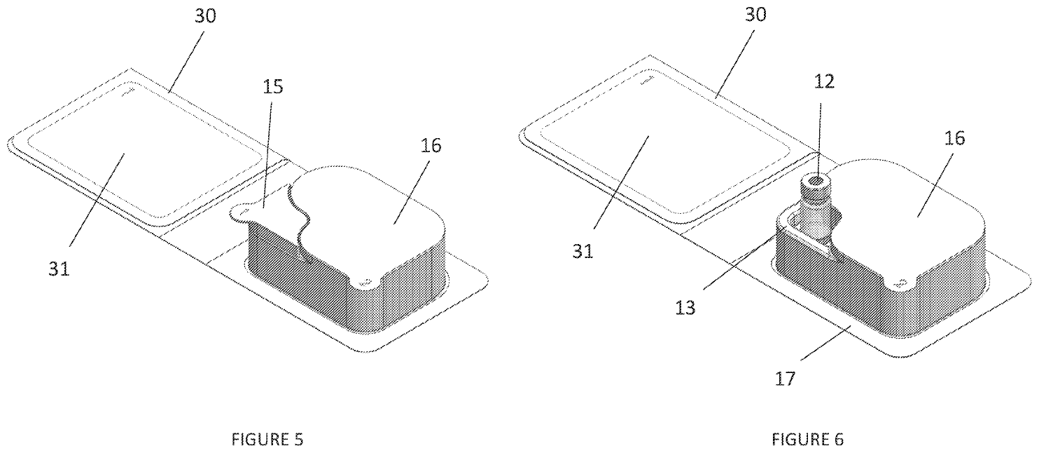

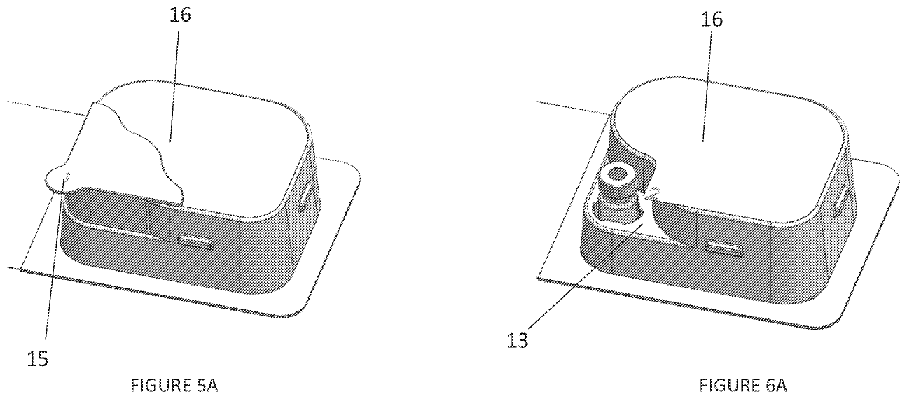

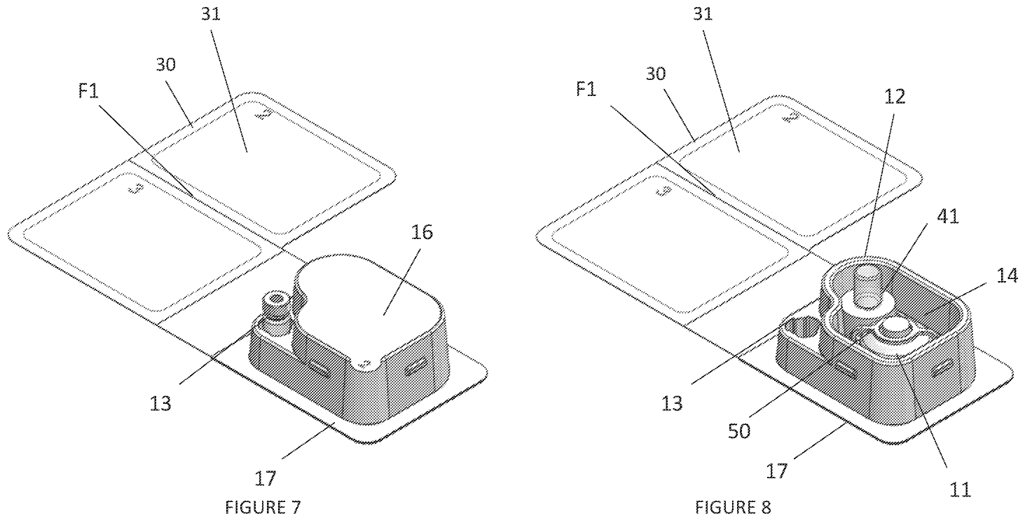

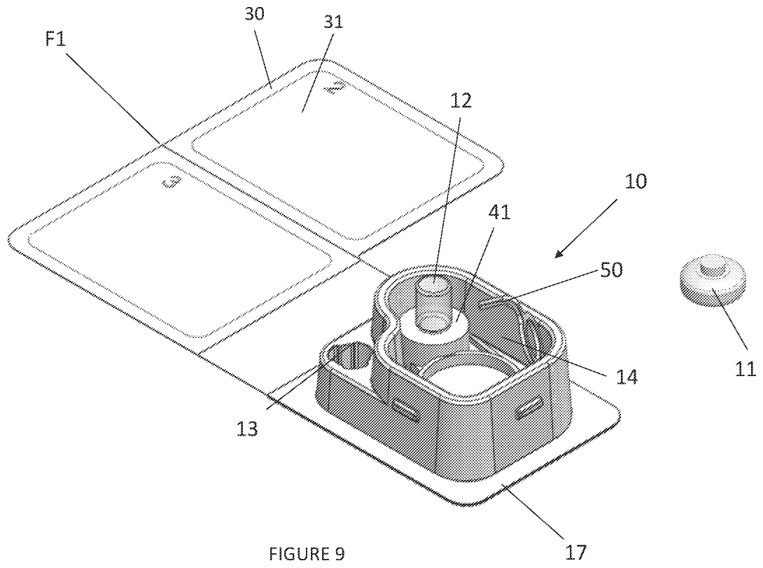

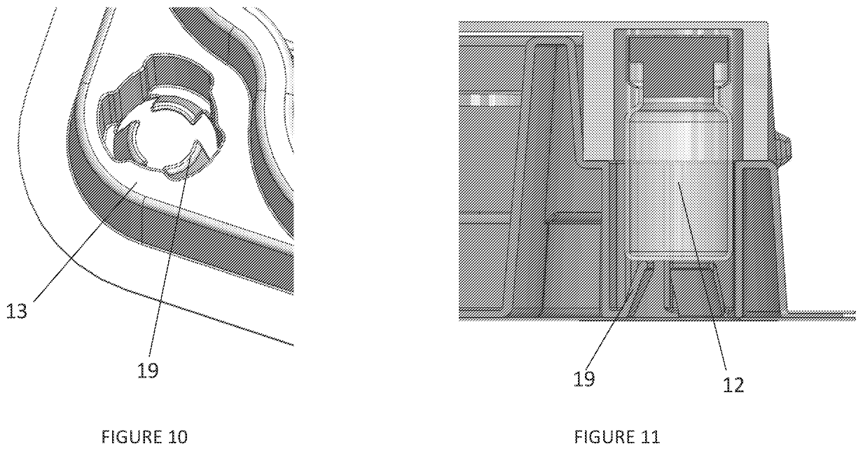

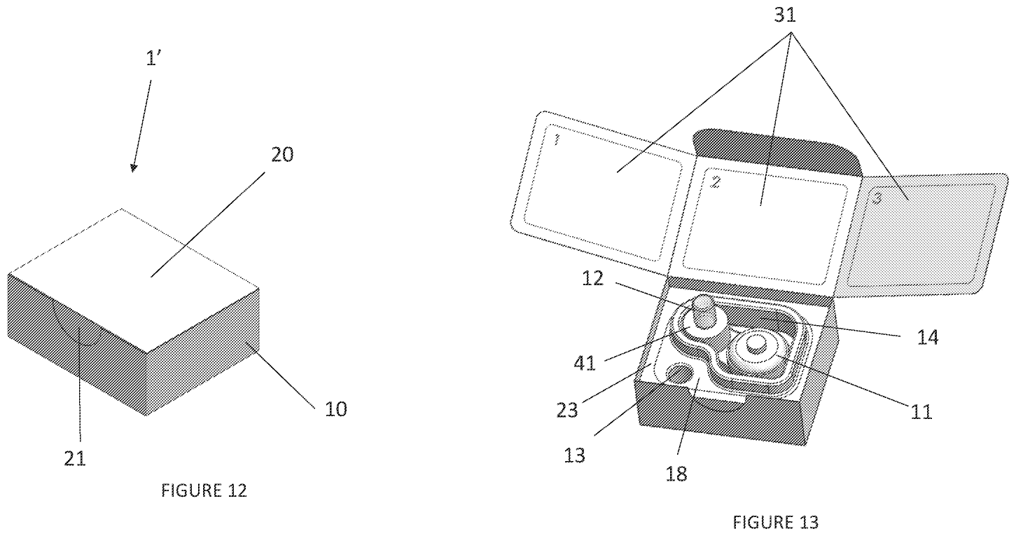

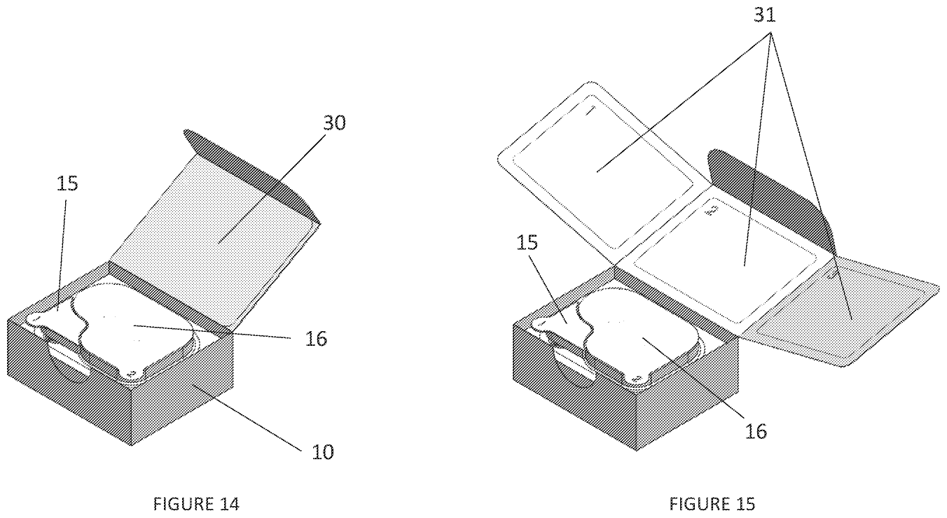

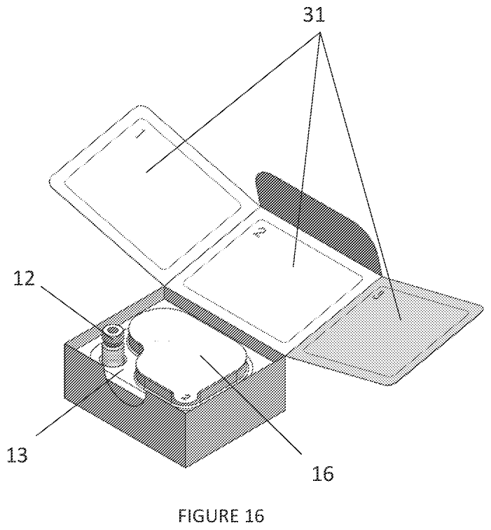

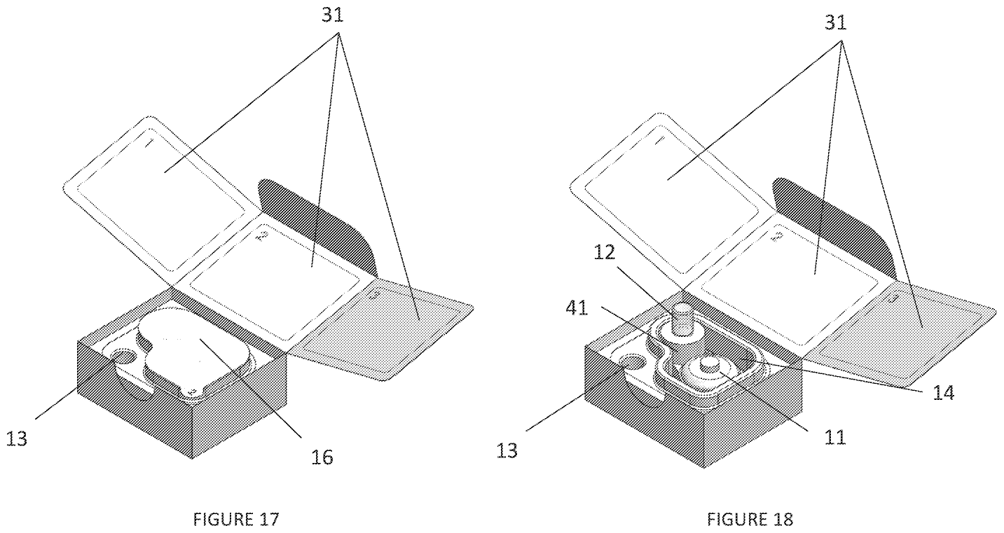

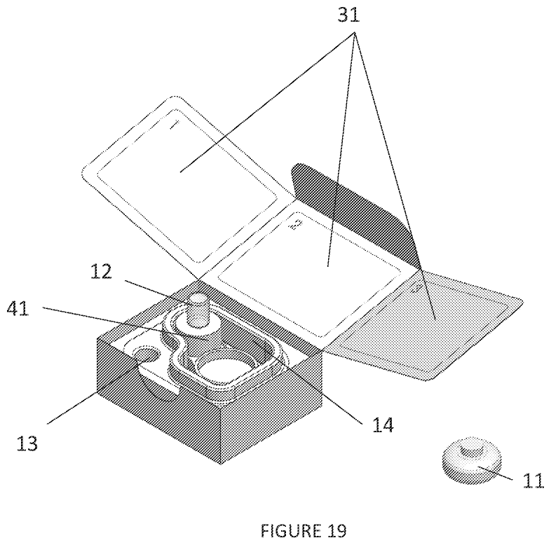

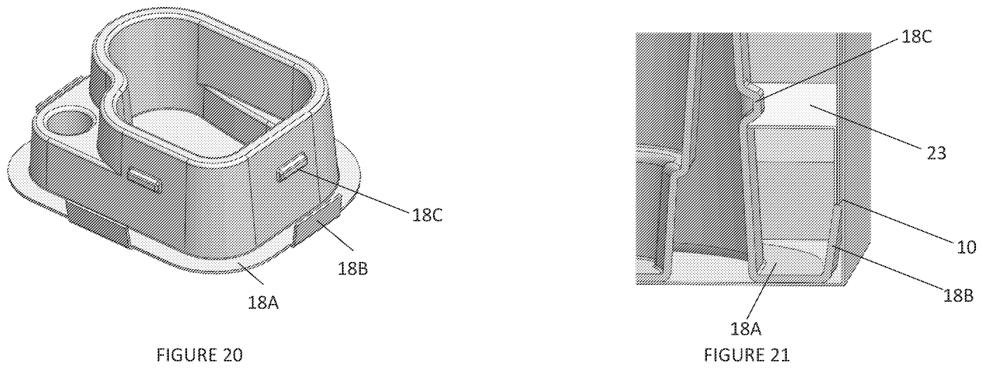

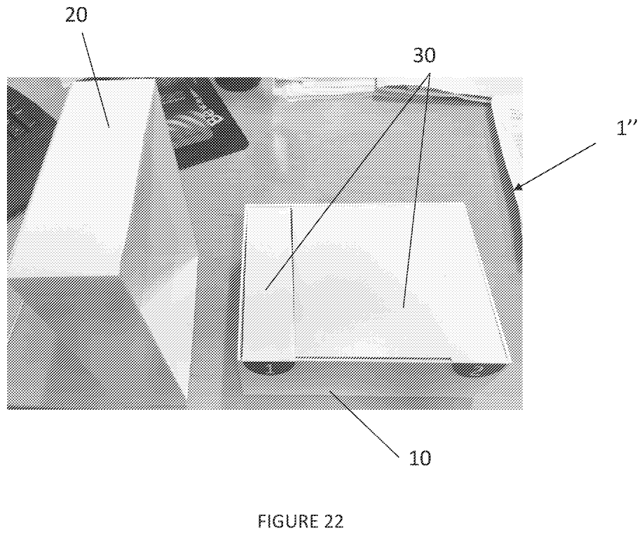

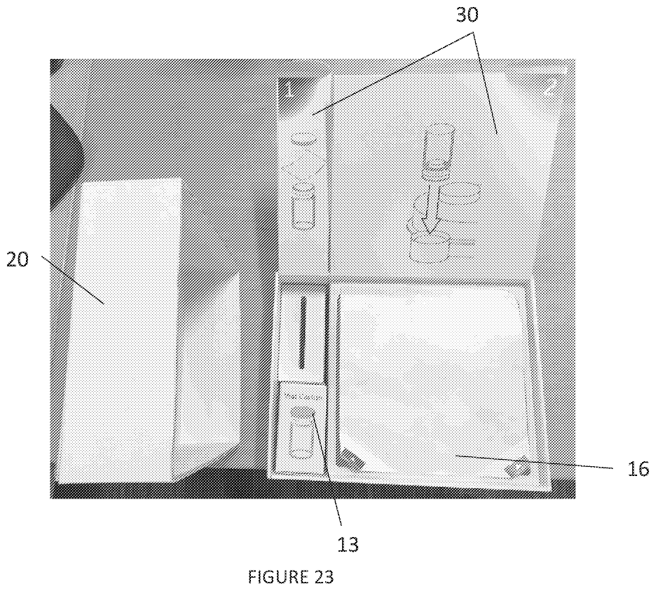

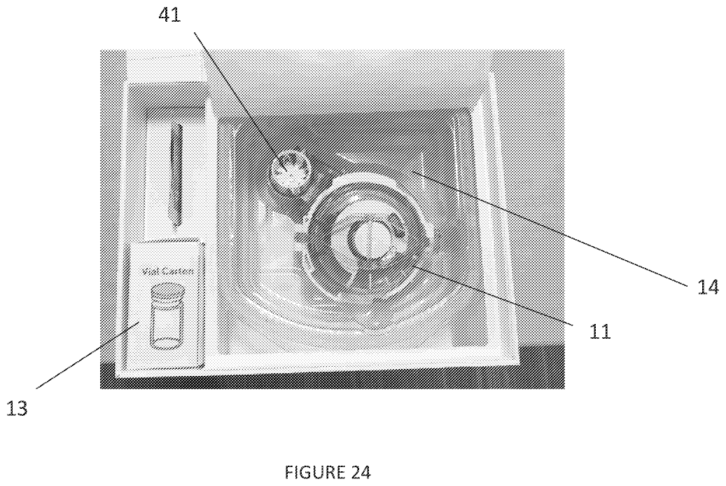

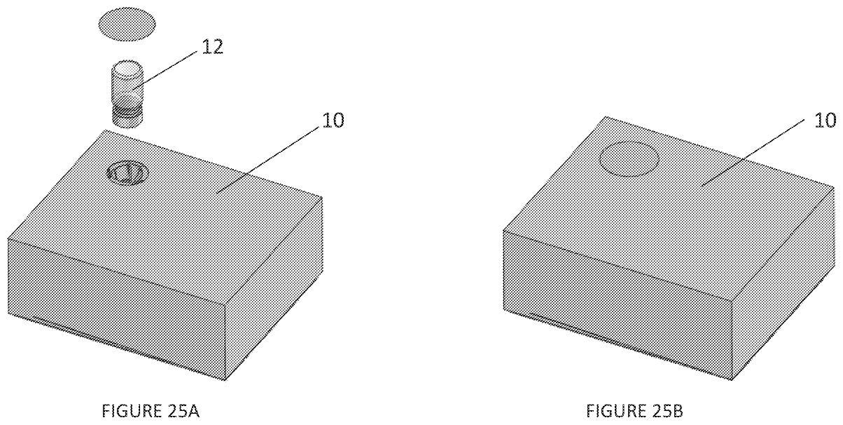

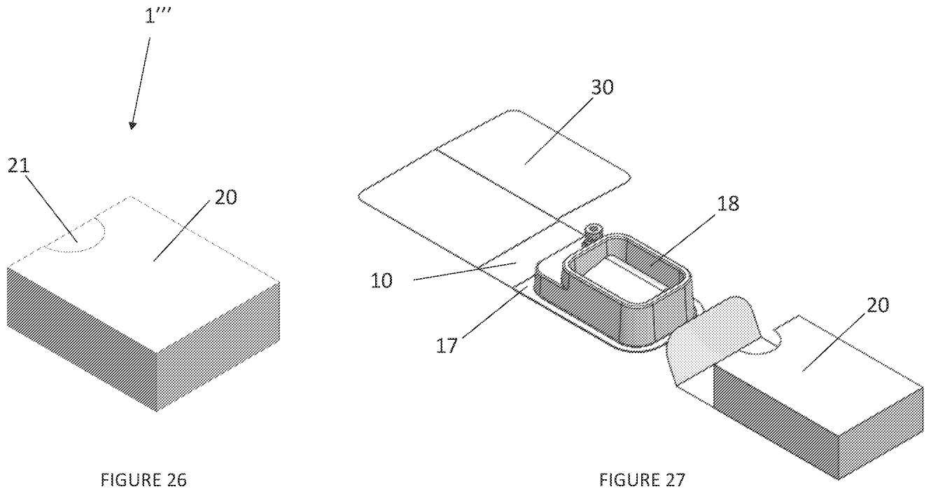

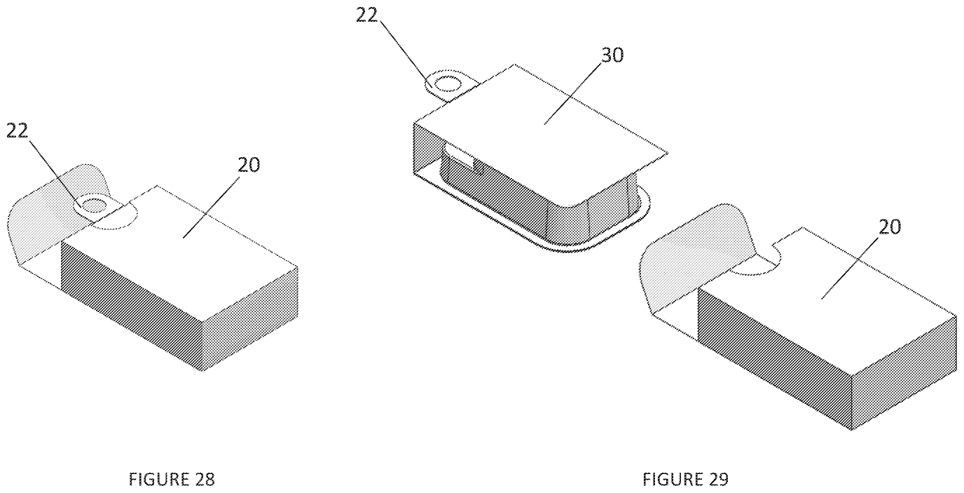

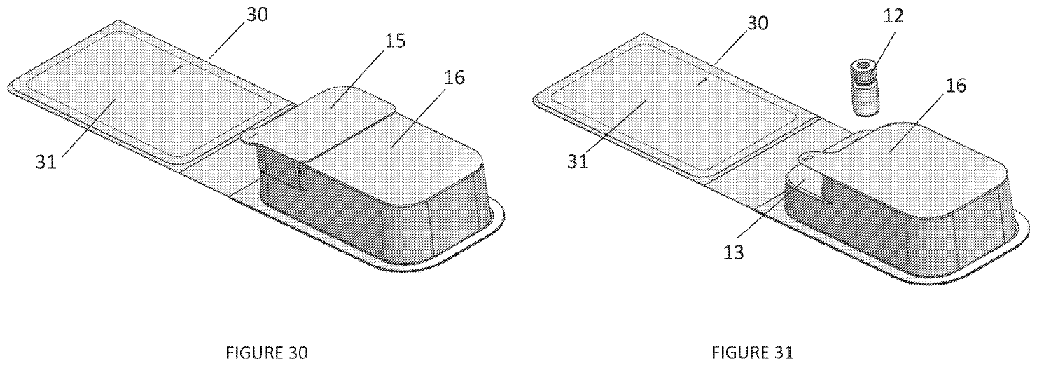

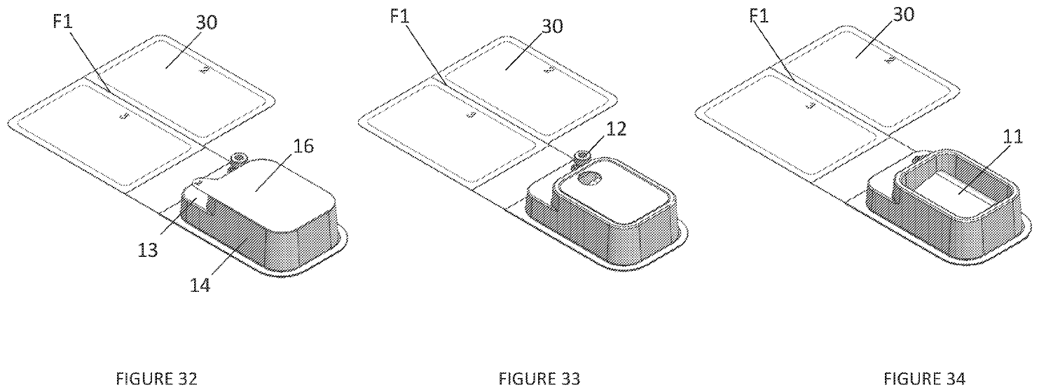

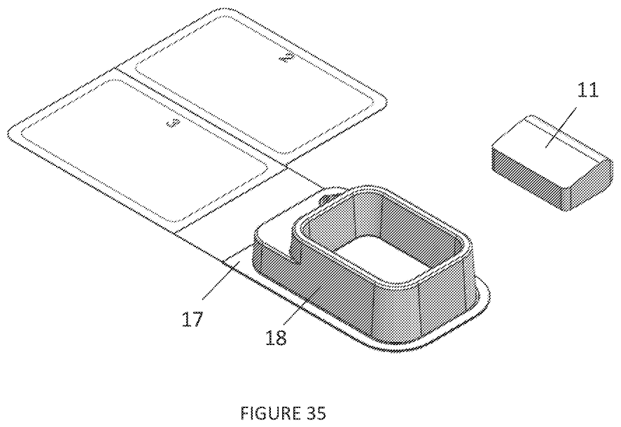

FIGS. 5A and 6A show an alternative embodiment;

FIG. 7 shows the foldable portion in a fully unfolded position;

FIG. 8 shows the second compartment open and the vial attached to the transfer mechanism;

FIG. 9 shows the packaging with the filled OBI removed for use;

FIG. 10 is a perspective view of a portion of the first compartment, showing the flexible tabs;

FIG. 11 is a cross-sectional view of the first compartment, showing the vial in place;

FIG. 12 is a perspective view of medical device packaging according to a second example embodiment of the present invention, showing the cover/lid in a closed position;

FIG. 13 shows the box with the cover/lid in an open position and with the foldable portion in a fully unfolded position;

FIG. 14 shows the box with the cover/lid in an open position and with the foldable portion in a folded position;

FIG. 15 shows the foldable portion in an unfolded position;

FIG. 16 shows the first compartment open;

FIG. 17 shows the first compartment open and the vial removed therefrom;

FIG. 18 shows the second compartment open and the vial attached to the transfer mechanism;

FIG. 19 shows the packaging with the filled OBI removed for use;

FIG. 20 is a perspective view of the tray;

FIG. 21 is a cross-sectional view of part of the tray in the box;

FIG. 22 is a view of medical device packaging according to a third example embodiment of the invention, showing the cover/sleeve in an open position and with the foldable portion in a folded position;

FIG. 23 shows the foldable portion in an unfolded position and the first compartment open;

FIG. 24 shows the second compartment open and the transfer mechanism ready to receive the vial;

FIGS. 25A and 25B show how the vial can be inserted after the packaging has been assembled;

FIG. 26 is a perspective view of medical device packaging according to a fourth example embodiment of the present invention, showing the packaging in a fully closed position;

FIG. 27 shows the box removed from the cover/outer box and with the foldable portion in a fully unfolded position;

FIG. 28 shows the cover/outer box still in a closed position;

FIG. 29 shows the cover/outer box in an open position and the box removed from the cover/outer box, with the foldable portion in a folded position;

FIG. 30 shows the foldable portion in a partially unfolded position;

FIG. 31 shows the first compartment open;

FIG. 32 shows the foldable portion in a fully unfolded position;

FIGS. 33 and 34 show the second compartment open giving access to the OBI;

FIG. 35 shows the packaging with the filled OBI removed for use;

### DETAILED DESCRIPTION

In the present disclosure, the following terms may be understood in view of the below explanations:

The term “on-body injector” or “OBI” may refer to a device intended for the injection of a medicament into the body wherein the device is attached to the body and actuated by the patient to automatically deliver a dose of medicament through the skin.

The term “cover” may refer to any type of cover which, when placed in a closed position on the box, prevents the compartments from being opened. Examples include a carton-style outer box, a removable sleeve open at one or both ends, a lid hinged to the box.

The term “instructions for use” or “IFU” may refer to any, all or part of the instructions for use and/or Quick Reference Guide of the device.

The term “Quick Reference Guide” or “QRG” may refer to a simplified, possibly visually depicted, version of the IFU.

### First Example Embodiment

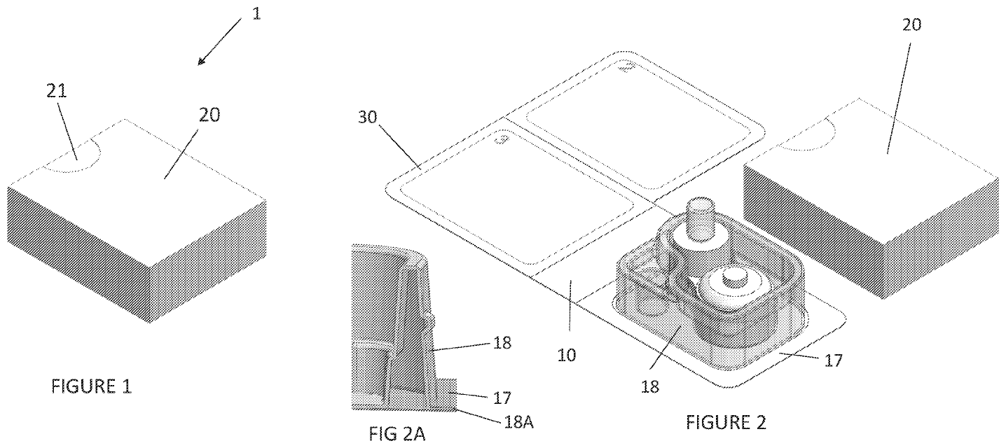

FIGS. 1-11 illustrate a first example embodiment of the claimed invention. Medical device packaging 1 comprises a box 10 containing a storage system for an OBI 11 and a medicament vial 12. The storage system comprises a moulded tray 18 including a first compartment 13 for storing the medicament vial 12 and a second compartment 14 for storing the OBI 11 and a medicament transfer mechanism 41. The moulded tray 18 is mounted on a laminated base card 17. The OBI 11 is retained in the box 10 by a retainer in the form of a clear plastic retention strap 50 which remains locked until the OBI 11 has been filled with medicament.

The box 10 is configured to be received within a cover 20 in the form of an outer box or removable sleeve, sealed with a tamper-proof sticker 21 (FIG. 1).

When the cover 20 has been removed, a foldable portion 30 of the box 10 can be unfolded (as will be described in more detail below) so that the packaging can lie fully flat, “placemat-style”, as shown in FIG. 2, The foldable portion 30 can depict instructions for use of the OBI divided into separate, sequential steps, as will be described below. The removed cover 20 can be placed in a convenient empty space defined by the unfolded foldable portion 30. Human factors studies show there is benefit in the user having the satisfaction of emptying the outer box and laying out the contents in front of them on a flat surface before starting to use the medical device.

As shown in FIG. 2A, the moulded tray 18 is adhered to the laminated base card 17, with a peripheral flange 18A of the tray 18 sandwiched between two layers of the base card 17.

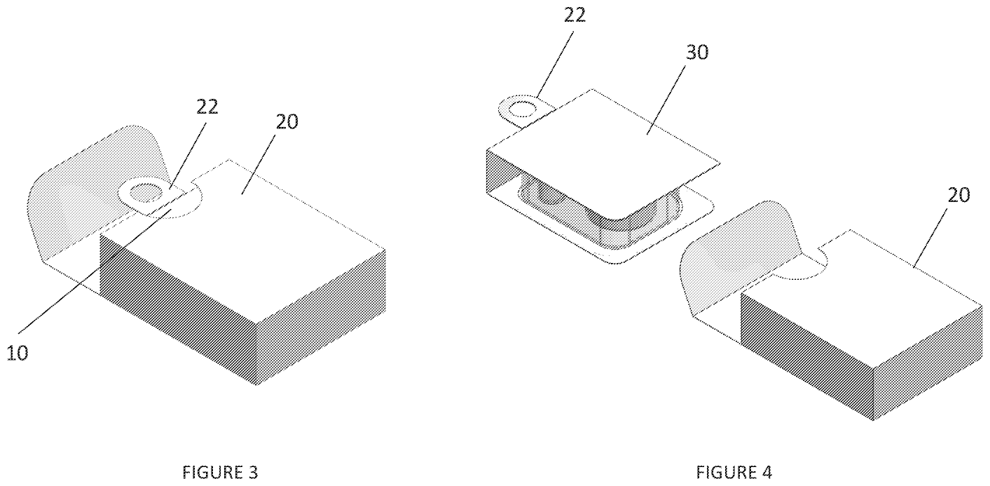

Further detail of the first example embodiment will now be described with reference to FIGS. 3-11.

After removal of the tamper-proof sticker 21, the outer box 20 can be opened to reveal a finger pull loop 22 which is attached to the box 10 (FIG. 3).

The finger pull loop 22 can be grasped in order to pull the box 10 out of the outer box 20. As shown in FIG. 4, upon removal from the box 20 the foldable portion 30 is in a fully folded position which covers the OBI 11 from view.

The foldable portion 30 can be moved to a partially unfolded position (FIG. 5) to reveal first and second compartments 13, 14 of the box, mounted on the laminated base card 17. Each of the compartments 13, 14 is sealed with a peelable lid 15, 16 for example made from Tyvek™. Alternatively, the compartments 13, 14 may be sealed with a removable lid made, for example, from rigid plastic. The partially unfolded position of FIG. 5 also reveals the first steps of the QRG (indicated with a “1” in FIG. 5) printed on a panel 31 of the foldable portion 30. These first steps are those associated with the first compartment 13 (also indicated with a “1” in FIG. 5). In the first unfolded position, only the first steps of the instructions for use may be visible (i.e., all subsequent steps may be hidden from the user's view).

The first steps of the QRG can include prompting the user to remove the peelable lid 15 from the first compartment 13 to reveal a medicament vial 12 therein (FIG. 6). The first steps can also prompt the user to remove the vial 12 from the first compartment 13.

In an alternative embodiment (illustrated in FIGS. 5A and 6A), the numbered part of the peelable lid 16 on the second compartment 14 is covered by the peelable lid 15 of the first compartment 13, only becoming visible and accessible after the first compartment 13 has been opened.

The foldable portion 30 can next be unfolded to a fully unfolded position (FIG. 7) in which second and third steps of the QRG are revealed (indicated with “2” and “3” in FIG. 7). The second steps of the QRG are printed on a panel 31 on the reverse of the foldable portion 30 containing the first steps and are only revealed when the foldable portion 30 is unfolded about fold line F1. The QRG steps are revealed in the order they are required, thus guiding the user through the steps and minimising user error.

The second steps of the QRG can prompt the user to remove the peelable lid 16 from the second compartment 14 to reveal the OBI 11 and a transfer mechanism 41 (FIG. 8). Neither the transfer mechanism 41 nor the OBI 11 can be removed from the box 10 at this stage. The OBI 11 is locked in place by the retention strap 50. The vial 12 can be attached to the transfer mechanism 41 in order to enable transfer of the medicament from the vial 12 to the transfer mechanism 41 and then into the OBI 11.

Once the OBI 11 is filled with medicament, the second and/or third steps can prompt actuating an unlocking mechanism, which releases retention strap 50 into an open position after which the OBI 11 can be removed from the box 10 and used (FIG. 9).

As shown in FIGS. 10 and 11, the vial 12 is retained in the first compartment 13 by three equispaced flexible tabs 19, for example, which flex outwardly into equispaced recesses as the vial 12 is first inserted into the compartment (from the bottom of FIG. 11) and then back into contact with the underside of the vial 12 to retain it until required. This one-way insertion of the vial 12 into the first compartment 13 from below enables, for example, a pharmacist to insert a cartridge or vial of medicament into the packaging ready for use without having to open the peelable lid 15. Other one-way insertion features may be contemplated. See for example FIGS. 25A and 25B and description thereof below.

### Second Example Embodiment

FIGS. 12-21 illustrate a second example embodiment of the claimed invention. Medical device packaging 1′ comprises a box 10 containing a storage system for an OBI 11 and a medicament vial 12. The storage system comprises a moulded tray 18 including a first compartment 13 for storing the medicament vial 12 and a second compartment 14 for storing the OBI 11 and a medicament transfer mechanism 41. The moulded tray 18 may be mounted on a laminated base card as described above then the tray 18 (and base card) are mounted in the box 10 with a box insert 23 to cover the empty space between the box and the edge of the tray. The OBI 11 may be retained in the box 10 by a retainer in the form of a clear plastic retention strap 50 (not illustrated) which remains locked and cannot be removed until the OBI 11 has been filled with medicament.

The box 10 is configured to be received within a cover 20 in the form of a hinged lid, sealed with a tamper-proof sticker 21 (FIG. 12).

The lid 20 can be opened to reveal a foldable portion 30 of the box 10 which can be unfolded (as will be described in more detail below) so that the entire QRG can be displayed simultaneously, as shown in FIG. 13.

Further detail of the second example embodiment will now be described with reference to FIGS. 14-21.

After removal of the tamper-proof sticker 21, the lid 20 can be opened to reveal first and second compartments 13, 14 of the box 10, each sealed with a peelable lid 15, 16 for example made from Tyvek™ (FIG. 14). Alternatively, the compartments 13, 14 may be sealed with a removable lid made, for example, from rigid plastic. A foldable portion 30 of the box 10 is visible, in a folded position. Optionally some steps of the QRG can be printed on the visible part of the foldable portion 30.

The foldable portion 30 can be unfolded to an unfolded position (FIG. 15) in which all of the steps of the QRG are visible, printed on numbered panels 31 of the foldable portion 30 (indicated with as “1” “2” and “3” in FIG. 15). Certain steps of the QRG correspond with similarly numbered first and second compartments 13, 14 (indicated with “1” and “2” in FIG. 15).

The first steps of the QRG can prompt the user to remove the peelable lid 15 from the first compartment 13 to reveal a medicament vial 12 therein (FIG. 16). The vial 12 can be removed from the first compartment 13 (FIG. 17).

The second steps of the QRG can prompt the user to remove the peelable lid 16 from the second compartment 14 to reveal the OBI 11 and a transfer mechanism 41 (FIG. 18). Neither the transfer mechanism 41 nor the OBI 11 can be removed from the box 10 at this stage. The vial 12 can be attached to the transfer mechanism 41 in order to transfer the medicament from the vial 12 to the transfer mechanism 41 and then into the OBI 11.

Once the OBI 11 is filled with medicament, an unlocking mechanism is actuated after which the OBI 11 can be removed from the box 10 and used (FIG. 19).

The vial 12 may be retained in the first compartment 13 in the manner shown in FIGS. 10 and 11.

The tray 18 may be retained in the box 10 as illustrated in FIGS. 20 and 21. The tray 18 has a peripheral flange 18A which sits in the bottom of the box 10. Integral snap features 18B extend upwardly from the flange 18A and can be hooked under a portion of the box wall (FIG. 21). Alternatively the peripheral flange and snap features can be provided on the laminated base card. The tray 18 is also provided with a plurality of externally-protruding bump off features 18C under which the box insert 23 can be retained.

### Third Example Embodiment

FIGS. 22-24 illustrate a third example embodiment of the claimed invention. Medical device packaging 1″ comprises a box 10 containing a storage system for an OBI 11 and a medicament vial 12. The storage system comprises moulded tray 18 including a first compartment 13 for storing the medicament vial 12 and a second compartment 14 for storing the OBI 11 and a medicament transfer mechanism 41. The moulded tray 18 is mounted on a laminated base card 17. The OBI 11 is retained in the box 10 by a retainer in the form of a clear plastic retention strap 50 which remains locked until the OBI 11 has been filled with medicament. The tray 18 may be retained in the box 10 as illustrated in FIGS. 20 and 21.

The box 10 is configured to be received within a cover 20 in the form of a removable sleeve, which may be sealed with a tamper-proof sticker (FIG. 22). As shown in FIG. 22, the sleeve 20 is removed to reveal a foldable portion 30 of the box 10 which can be unfolded (as will be described in more detail below) so that the entire QRG can be displayed simultaneously, as shown in FIG. 23. The foldable portion 30 is labelled with the digits “1” and “2” to indicate to the user the order in which they should be unfolded. Optionally (not illustrated) some steps of the QRG could be printed on the visible outside part of the foldable portion 30.

The foldable portion 30 can be unfolded to an unfolded position (FIG. 23) in which all of the steps of the QRG are visible, printed on numbered panels 31 of the foldable portion 30 (indicated with as “1” and “2” in FIG. 23). These steps of the QRG correspond with similarly numbered first and second compartments 13, 14. The numbered panels are independently openable. In this way the user is directed to first unfold panel “1” to read the QRG steps associated with the first compartment 13. Once these steps are complete, the user is directed to unfold panel “2” to read the QRG steps associated with the second compartment 14.

The first compartment 13 contains a medicament vial in a suitably labelled vial carton and a slot in which, for example, a disinfecting wipe could be provided.

Generally, the medicament vial is required to be stored in a temperature controlled environment, whereas the OBI is not. Therefore, there is a practical advantage for the supplier to be able to insert the medicament vial carton into the first compartment 13 after the remainder of the packaging (with the exception of the cover sleeve 20) has been assembled. To this end, the first compartment 13 may optionally be provided with an opening in the side or base thereof, through which the medicament vial, cartridge or vial carton can be inserted without affecting the foldable portion 30. An example of this is illustrated in FIGS. 25A and 25B.

The second compartment 14, sealed with a peelable and removable Tyvek™ lid, contains the OBI 11 and a transfer mechanism 41. Neither the transfer mechanism 41 nor the OBI 11 can be removed from the box 10 at this stage. The vial 12 can be attached to the transfer mechanism 41 in order to transfer the medicament from the vial 12 to the transfer mechanism 41 and then into the OBI 11.

Once the OBI 11 is filled with medicament, an unlocking mechanism is actuated after which the OBI 11 can be removed from the box 10 and used.

### Fourth Example Embodiment

FIGS. 26-35 illustrate a fourth example embodiment of the claimed invention, adapted for a different medical OBI device where there is no transfer mechanism. Medical device packaging 1′″ comprises a box 10 containing a storage system for an OBI 11 and a medicament vial or cartridge 12. The storage system comprises a moulded tray 18 including a first compartment 13 for storing the medicament vial 12 and a second compartment 14 for storing the OBI 11. The moulded tray 18 is mounted on a laminated base card 17.

The box 10 is configured to be received by a cover 20 in the form of an outer box or removable sleeve, sealed with a tamper-proof sticker 21 (FIG. 26).

When the cover 20 has been removed, a foldable portion 30 of the box 10 can be unfolded (as will be described in more detail below) so that the packaging can lie fully flat, “placemat-style”, as shown in FIG. 27. The removed cover 20 can be placed in a convenient empty space defined by the unfolded foldable portion 30. Human factors studies show there is benefit in the user having the satisfaction of emptying the outer box and laying out the contents in front of them on a flat surface before starting to use the medical device.

The moulded tray 18 may be adhered to the laminated base card 17 as illustrated in FIG. 2A.

Further detail of the first example embodiment will now be described with reference to FIGS. 28-35.

After removal of the tamper-proof sticker 21, the outer box 20 can be opened to reveal a finger pull loop 22 which is attached to the box 10 (FIG. 28).

The finger pull loop 22 can be grasped in order to pull the box 10 out of the outer box 20. As shown in FIG. 29, the foldable portion 30 is in a fully folded position which covers the OBI 11 from view.

The foldable portion 30 can be moved to a partially unfolded position (FIG. 30) to reveal first and second compartments 13, 14 of the box, mounted on the laminated base card 17. Each of the compartments 13, 14 is sealed with a peelable lid 15, 16 for example made from Tyvek™. Alternatively, the compartments 13, 14 may be sealed with a removable lid made, for example, from rigid plastic. The partially unfolded position of FIG. 30 also reveals the first steps of the QRG (indicated with a “1” in FIG. 30) printed on a panel 31 of the foldable portion 30. These first steps are those associated with the first compartment 13 (also indicated with a “1” in FIG. 30).

Having read the first steps of the QRG, the peelable lid 15 can be removed from the first compartment 13 to reveal a medicament vial 12 therein (FIG. 31). The vial 12 can be removed from the first compartment 13.

The numbered part of the peelable lid 16 on the second compartment is covered by the peelable lid 15 of the first compartment 13, only becoming visible and accessible after the first compartment 13 has been opened.

The foldable portion 30 can next be unfolded to a fully unfolded position (FIG. 32) in which second and third steps of the QRG are revealed (indicated with “2” and “3” in FIG. 32). The second steps of the QRG are printed on a panel 31 on the reverse of the foldable portion 30 containing the first steps and are only revealed when the foldable portion 30 is unfolded about fold line F1. The QRG steps are revealed in the order they are required, thus guiding the user through the steps and minimising user error.

Having read the second steps of the QRG, the peelable lid 16 can be removed from the second compartment 14 to reveal a device cover (FIG. 33) which is removed to reveal the OBI 11 (FIG. 34). The vial 12 is then assembled into the OBI 11 so that the device is ready to use (FIG. 35).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCE NUMERALS

1, 1′, 1″, 1″″ medical device packaging
10 box
11 on-body injector (“OBI”)
12 medicament vial
13 first compartment
14 second compartment
15 first peelable lid
16 second peelable lid
17 laminated base card
18 moulded tray
18A peripheral flange of tray
18B integral snap features of tray
18C bump off features of tray
19 flexible tabs
20 cover
21 tamper-proof sticker
22 finger pull loop
23 box insert
30 foldable portion
31 panel
F1 fold line
41 transfer mechanism
50 retention strap

What is claimed is:

1. Medical device packaging comprising:
a box comprising a first compartment and a second compartment, the first and second compartments being independently openable to reveal contents of the first and second compartments respectively;
a cover moveable between a closed position and an open position and which, when placed in the closed position on the box, prevents the first and second compartments from being opened;
instructions for use of the device provided on a foldable portion of the box, the instructions for use only being fully visible when the cover is placed in the open position and the foldable portion is moved from a folded to an unfolded position,
wherein the first compartment is configured for storing a medicament vial or cartridge and the second compartment is configured for storing an injection device which may be an on body injector.

2. The medical device packaging of claim 1, wherein said foldable portion comprises at least two panels which are moveable from the folded position to the unfolded position in a sequence such that steps of the instructions for use provided on each panel are revealed in a desired order.

3. The medical device packaging of claim 2, wherein said sequence includes opening said first compartment after moving one of said at least two panels from the folded position to the unfolded position.

4. The medical device packaging of claim 2, wherein said sequence includes opening said second compartment after moving a second one of said at least two panels from the folded position to the unfolded position.

5. The medical device packaging of claim 1, wherein said foldable portion has an inner and an outer surface and wherein at least one of said panels is provided on each of said inner and outer surfaces.

6. The medical device packaging of claim 1, wherein said instructions for use are printed directly onto said foldable portion.

7. The medical device packaging of claim 1, wherein said cover is a lid, and wherein the lid is moveable between said closed position and said open position.

8. The medical device packaging of claim 1, wherein said cover comprises said foldable portion.

9. The medical device packaging of claim 1 wherein said cover is a removable sleeve or outer box, and wherein the cover is slidable over said first and second compartments between said open position and said closed position.

10. The medical device packaging of claim 9, wherein said box comprises a pull tab or pull loop to facilitate removal of the box from the removable sleeve or outer box.

11. The medical device packaging of claim 9, wherein, when the foldable portion is in the unfolded position, the foldable portion can be laid flat.

12. The medical device packaging of claim 1, wherein one or both of said first and second compartments comprises a removable or peelable cover, and wherein said at least one of said first and second compartments are independently openable by removal of said removable or peelable cover(s).

13. The medical device packaging claim 12, wherein said first compartment further comprises an opening through which a medicament vial or cartridge can be inserted into said first compartment without opening said removable or peelable cover.

14. A storage system for storing a medical device, the storage system comprising
a base;
a tray mounted on the base and for receiving the medical device, and
a retainer for releasably retaining the medical device in the tray,
wherein the retainer is moveable between a locked position in which the medical device cannot be removed from the tray and an unlocked position in which the medical device can be removed from the tray.

15. The storage system of claim 14, further comprising a transfer mechanism capable of transferring medicament from a medicament vial or cartridge to the medical device, wherein the transfer mechanism comprises an unlocking mechanism which is actuable upon completion of medicament transfer to move the retainer from the locked to the unlocked position.

16. The storage system of claim 14, wherein the retainer is hingedly connected to the tray and, wherein the transfer mechanism is permanently attached to the tray.

17. The storage system of claim 14, wherein the tray comprises a peripheral flange that is glued or otherwise permanently attached to the base.

18. The storage system of claim 17, wherein the peripheral flange is located between two layers of the base so that, in use, the tray cannot be removed from the base.
